(19) United States
(12) Patent Application Publication
(10) Pub. No.: US 2007/0073323 A1
(43) Pub. Date: Mar. 29, 2007

(54) Apparatus for manipulating and fastening stomach tissue to treat gastroesophageal reflux disease

(75) Inventors: Brett J. Carter, Monroe, WA (US); John M. Adams, Sammamish, WA (US)

Correspondence Address: Richard O. Gray, Jr., Suite 350, 155-108th Avenue N.E., Bellevue, WA, 98004-5973, US

(73) Assignee: EndoGastric Solutions, Inc.

(21) Appl. No.: 11/342,292

(22) Filed: Jan. 27, 2006

Related U.S. Application Data
(63) Continuation-in-part of application No. 11/235,668, filed on Sep. 26, 2005.

(51) Int. Cl. A61B 17/08

(52) U.S. Cl. 606/153

(57) ABSTRACT

A transoral gastroesophageal flap valve (GEFV) restoration device presents stomach tissue for fastening. The device comprises a longitudinal member having a portion arranged for transoral placement into a stomach. A tissue shaper is carried on the distal end of the longitudinal member that shapes stomach tissue within the stomach into a GEFV. The tissue shaper comprises a pair of hingedly coupled first and second arms for receiving the stomach tissue there between. The device further comprises a fastener director that directs a fastener into the stomach tissue along a path that is devoid of any deviations or bends greater than 45 degrees.

### RELATED APPLICATION DATA

The present patent application is a continuation-in-part application of U.S. patent application Ser. No. 11/235,668, filed Sep. 26, 2005.

### FIELD OF THE INVENTION

The present invention generally relates to manipulation of stomach tissue as by folding or molding and fastening to treat gastroesophageal reflux disease. The present invention more particularly relates to positioning tissue for fixation with devices in the stomach to promote reliable fixating of stomach tissue.

### BACKGROUND

Gastroesophageal reflux disease (GERD) is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV gastroesophageal flap. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laparoscopic surgical procedures are available for treating GERD. One surgical approach is the Nissen fundoplication. The Nissen approach typically involves a 360-degree wrap of the fundus around the gastroesophageal junction. The procedure has a high incidence of postoperative complications. The Nissen approach creates a 360-degree moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction. Another surgical approach is the Hill repair. In the Hill repair, the gastroesophageal junction is anchored to the posterior abdominal areas, and a 180-degree valve is created by a system of sutures. The Hill procedure restores the moveable flap, the cardiac notch and the Angle of His. However, all of these surgical procedures are very invasive, regardless of whether done as a laparoscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak, in part due to the fact, that the esophagus is not covered by serosa, a layer of very sturdy, yet very thin tissue, covering and stabilizing all intraabdominal organs, similar like a fascia covering and stabilizing muscle. Involvement of esophageal tissue in the repair of a gastroesophageal flap valve poses unnecessary risks to the patient, such as an increased risk of fistulas between the esophagus and the stomach.

A new and improved apparatus and method for restoration of a gastroesophageal flap valve is fully disclosed in U.S. Pat. No. 6,790,214, issued Sep. 14, 2004, is assigned to the assignee of this invention, and is incorporated herein by reference. That apparatus and method provides a transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

Whenever tissue is to be maintained in a shape as, for example, in the improved assembly last mentioned above, it is necessary to first grip stomach tissue and then fasten at least two layers of gripped tissue together. In applications such as gastroesophageal flap valve restoration, it is desirable to grip stomach tissue displaced from the esophageal opening into the stomach so that when the stomach tissue is pulled aborally to form a flap, the flap will have sufficient length to cover the opening and function as a flap valve. With the gastroesophageal anatomy thus restored, the GERD will be effectively treated.

Deploying fasteners through the flap forming stomach tissue is not a simple matter. The formed flap is generally parallel to the center axis of the longitudinal member. Hence, to deploy a fastener to maintain the flap, a fastener must be driven into the tissue layers substantially perpendicular to the tissue layers and hence perpendicular to the longitudinal member center axis. This requires a nearly ninety degree (90°) bend in the fastener driver. Such bends dramatically reduce the drive force which may be imparted to a fastener making deployment more difficult.

Hence, there is a need in the art for techniques and devices which enable more ready manipulation and fastening of stomach tissue from within the stomach. The present invention addresses these and other issues.

### SUMMARY

The invention provides a transoral gastroesophageal flap valve restoration device. The device comprises a longitudinal member, a portion of which is arranged for transoral placement into a stomach, and having a longitudinal axis. The device further comprises a tissue shaper carried on the longitudinal member that shapes stomach tissue into a shape, and a fastener director that directs a fastener into the stomach. The fastener director is devoid of any deviation greater than 45 degrees.

The fastener director may comprise a guide lumen that extends from the longitudinal member to the tissue shaper. The tissue shaper may comprise a pair of hingedly coupled first and second arms for receiving the stomach tissue there between, the second arm including an opening permitting the fastener to be driven through the stomach tissue while being held between the first and second arms. The second arm may have a distal end and the opening may be at the distal end of the second arm.

The first arm may include a wall generally transverse to the fastener director and the fastener director may direct the fastener through the wall. The guide lumen, within the longitudinal member, communicates with the wall. The distal end of the second member is closely adjacent the wall with the opening aligned with the lumen when a fastener is directed through the stomach tissue.

The device may comprise a plurality of fastener directors, each fastener director being arranged to direct a fastener into the stomach tissue along a path that is devoid of any deviation greater than 45 degrees. Each fastener director may comprise a guide lumen that extends through the longitudinal member to the tissue shaper.

The invention further provides a transoral gastroesophageal flap valve restoration device comprising a longitudinal member having a longitudinal axis and a portion arranged for transoral placement into a stomach and a tissue shaper carried on the longitudinal member that shapes stomach tissue into a shape. The tissue shaper comprises a pair of hingedly coupled first and second arms for receiving the stomach tissue there between. The device further comprises a fastener director that directs a fastener into the stomach tissue along a path that is devoid of any deviation greater than 45 degrees.

The invention further provides an assembly for transoral gastroesophageal flap valve restoration. The assembly comprises a longitudinal member, a portion of which is arranged for transoral placement into a stomach. The longitudinal member also has a longitudinal axis. The assembly further comprises a tissue shaper carried on the longitudinal member that shapes stomach tissue into a shape, a fastener that maintains the stomach tissue in the shape, and a fastener director that directs the fastener into the stomach tissue along a path that is devoid of any deviation greater than 45 degrees.

The fastener director may comprise a guide lumen that extends from the longitudinal member to the tissue shaper. The assembly may further comprise a deployment wire arranged to slidingly receive the fastener and guide the fastener down the guide lumen and into the stomach tissue. The assembly may further comprise a pusher that pushes the fastener along the deployment wire down the guide lumen.

The fastener may comprise first and second members having first and second ends, and a connecting member fixed to each of the first and second members intermediate the first and second ends and extending between the first and second members. The first and second members are separated by the connecting member, and one of the first and second members has a through channel for being received on the deployment wire.

The tissue shaper may comprise a pair of hingedly coupled first and second arms for receiving the stomach tissue there between, the second arm including an opening permitting the fastener to be driven through the stomach tissue while being held between the first and second arms. The second arm may have a distal end and the opening may be at the distal end of the second arm.

The first arm may include a wall generally transverse to the fastener director and the fastener director may direct the fastener through the wall. The guide lumen, within the longitudinal member, communicates with the wall. The distal end of the second member is closely adjacent the wall with the opening aligned with the lumen when a fastener is directed through the stomach tissue.

The assembly may comprise a plurality of fastener directors, each fastener director being arranged to direct a fastener into the stomach tissue along a path that is devoid of any deviation greater than 45 degrees. Each fastener director may comprise a guide lumen that extends through the longitudinal member to the tissue shaper.

According to another embodiment, a transoral gastroesophageal flap valve restoration device comprises a longitudinal member, a portion of which is arranged for transoral placement into a stomach, a tissue shaper carried on the longitudinal member that shapes stomach tissue into a shape, and a fastener director that directs a fastener along a fastener deployment wire and a path into the stomach tissue. The fastener deployment wire has a distal tip end and the tissue shaper is configured to receive and enclose the deployment wire tip end.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a front cross-sectional view of the esophageal-gastro-intestinal tract from a lower portion of the esophagus to the duodenum;

FIG. 2 is a front cross-sectional view of the esophageal-gastro-intestinal tract illustrating a Grade I normal appearance movable flap of the gastroesophageal flap valve (in dashed lines) and a Grade III reflux appearance gastroesophageal flap of the gastroesophageal flap valve (in solid lines);

FIG. 3 is a side view of an apparatus for restoring the flap of a GEFV according to an embodiment of the invention;

FIG. 4 is a view of the apparatus of FIG. 3 according to an embodiment of the invention being used to mold stomach tissue into a GEFV and to prepare the molded tissue for fixation;

FIG. 5 is a perspective view illustrating a manner in which the device of FIGS. 3 and 4 may deploy a fastener through molded stomach tissue layers;

FIG. 6 is a perspective view showing a fastener fully deployed;

FIG. 7 is a partial side view of an apparatus according to another embodiment; and

FIG. 8 is a side view of another apparatus according to a further embodiment.

### DETAILED DESCRIPTION

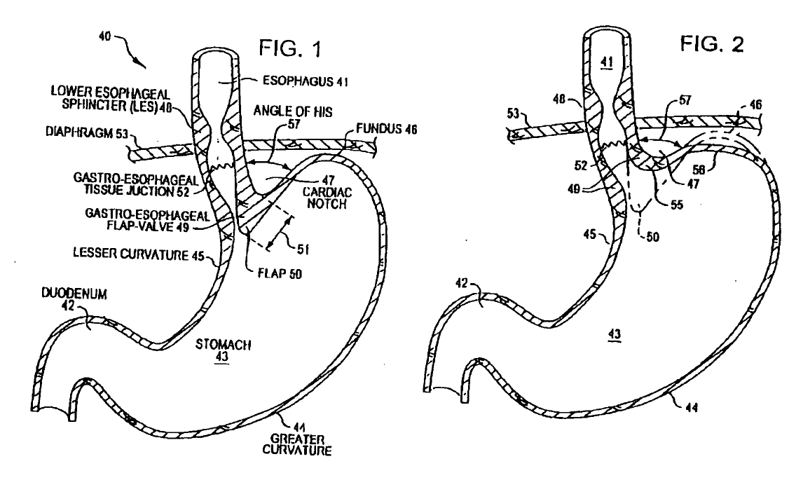

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue.

The fundus 46 of the greater curvature 44 forms the superior portion of the stomach 43, and traps gas and air bubbles for burping. The esophageal tract 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion.

The moveable portion of the GEFV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43. It is about 4 to 5 cm long (51) at it longest portion, and its length may taper at its anterior and posterior ends.

The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES) in the neck near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

FIG. 2 is a front cross-sectional view of an esophageal-gastro-intestinal tract illustrating a Grade I normal appearance movable flap 50 of the GEFV 49 (shown in dashed lines) and a deteriorated Grade III gastroesophageal flap 55 of the GEFV 49 (shown in solid lines). As previously mentioned, a principal reason for regurgitation associated with GERD is the mechanical failure of the deteriorated (or reflux appearance) gastroesophageal flap 55 of the GEFV 49 to close and seal against the higher pressure in the stomach. Due to reasons including lifestyle, a Grade I normal gastroesophageal flap 50 of the GEFV 49 may deteriorate into a Grade III deteriorated gastroesophageal flap 55. The anatomical results of the deterioration include moving a portion of the esophagus 41 that includes the gastroesophageal junction 52 and LES 48 toward the mouth, straightening of the cardiac notch 47, and increasing the Angle of His 57. This effectively reshapes the anatomy aboral of the gastroesophageal junction 52 and forms a flattened fundus 56.

The deteriorated gastroesophageal flap 55 shown in FIG. 2 has a gastroesophageal flap valve 49 and cardiac notch 47 that are both significantly degraded. Dr. Hill and colleagues developed a grading system to describe the appearance of the GEFV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEFV 49 illustrates a Grade I flap valve that is the least likely to experience reflux. The deteriorated gastroesophageal flap 55 of the GEFV 49 illustrates a Grade III (almost Grade IV) flap valve. A Grade IV flap valve is the most likely to experience reflux. Grades II and III reflect intermediate grades of deterioration and, as in the case of III, a high likelihood of experiencing reflux. With the deteriorated GEFV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently is a device, assembly, and method which may be employed to advantage according to an embodiment of the invention in restoring the normal gastroesophageal flap valve anatomy.

Referring now to FIG. 3, it shows a device 100 according to an embodiment of the present invention. The device 100 includes a longitudinal member 102 for transoral placement of the device 100 into the stomach. The longitudinal member 102 has a longitudinal axis 103. The device further includes a first arm 104, hereinafter referred to as the chassis, and a second arm 106, hereinafter referred to as the bail. The chassis 104 and bail are hingedly coupled at 107. The chassis 104 and bail 106 form a tissue shaper which, as described subsequently in accordance with this embodiment of the present invention, shapes tissue of the stomach into the flap of a restored gastroesophageal flap valve that is substantial parallel to the axis 103. The chassis 104 and bail 106 are carried at the distal end of the longitudinal member 102 for placement in the stomach.

The device 100 has a longitudinal passage (not shown) to permit an endoscope 110 to be guided through the device and into the stomach. Such a passage may be seen, for example, in copending application Ser. No. 11/172,427, filed Jun. 29, 2005, for APPARATUS AND METHOD FOR MANIPULATING STOMACH TISSUE AND TREATING GASTROESOPHAGEAL REFLUX DISEASE, and which application is incorporated herein by reference. This permits the endoscope to service as a guide for guiding the device 100 through the patient's throat, down the esophagus, and into the stomach. It also permits the gastroesophageal flap valve restoration procedure to be viewed at each stage of the procedure.

As will be seen subsequently, to facilitate shaping of the stomach tissue, the stomach tissue is drawn in between the chassis 104 and the bail 106. Further, to enable a flap of sufficient length to be formed to function as the flap of a gastroesophageal flap valve, the stomach tissue is pulled down so that the fold line is substantially juxtaposed to the opening of the esophagus into the stomach. Hence, the stomach is preferably first gripped at a point out and away from the esophagus and the grip point is pulled to almost the hinged connection 107 of the chassis 104 and bail 106. As described in copending application Ser. No. 11/001,666, filed Nov. 30, 2004, entitled FLEXIBLE TRANSORAL ENDOSCOPIC GASTROESOPHAGEAL FLAP VALVE RESTORATION DEVICE AND METHOD, which application is incorporated herein by reference, the device 100 is fed down the esophagus with the bail 106 substantially in line with the chassis 104. To negotiate the bend of the throat, and as described in the aforementioned referenced application, the chassis 104 and bail 106 are rendered flexible. The chassis 104 is rendered flexible by the slots 108 and the bail 106 is rendered flexible by the hingedly coupled links 112 including a distal link 109. Further details concerning the flexibility of the chassis 104 and the bail 106 may be found in the aforementioned referenced application.

The device further includes a tissue gripper (not shown). The gripper, as shown and described in the aforementioned copending application Ser. No. 11/172,427, comprises a helical coil. The coil is carried at the end of a cable (not shown) and may be attached to the end of the cable or be formed from the cable.

With continued reference to FIG. 3, the device 100 further comprises a fastener director 140. The fastener director includes a plurality of fastener deployment guides 142. Each fastener deployment guide 142 takes the form of a guide lumen. The guide lumens extend through the longitudinal member 102 to the first member 104 and terminate at delivery points 144 where a fastener is driven into the molded stomach tissue. The delivery ports 144 are within a wall 143 of the first member that is transverse to the longitudinal axis 103 of the longitudinal member 102 and the fastener director lumens 142.

The device 100 further includes a window 130 within the chassis 104. The window is formed of a transparent or semi-transparent material. This permits gastroesophageal anatomy, and more importantly the gastroesophageal junction (Z-line) to be viewed with the endoscope. The window includes a location marker 132 which has a know position relative to the fastener delivery points 144. Hence, by aligning the marker with a known anatomical structure, the fastener will be delivered a known distance from or at a location having a predetermined relation to the marker. For example, by aligning the marker with the Z-line, it will be know that the fastener will be placed aboral of the Z-line and that serosa tissue will be fastened to serosa tissue. As previously mentioned, this has many attendant benefits.

It may also be mentioned at this point that the device 100 further includes an invaginator 145 including a plurality of orifices 146. These orifices 146, which alternatively may be employed on the longitudinal member 102, are used to pull a vacuum to cause the device 100 to grip the inner surface of the esophagus. This serves to stabilize the esophagus and maintains device positioning during the procedure. This vacuum gripping of the esophagus may also be used to particular advantage if the patient suffers from a hiatal hernia. Upon being thus gripped, the esophagus may be moved downwardly with the device toward the stomach to eliminate the hiatal hernia.

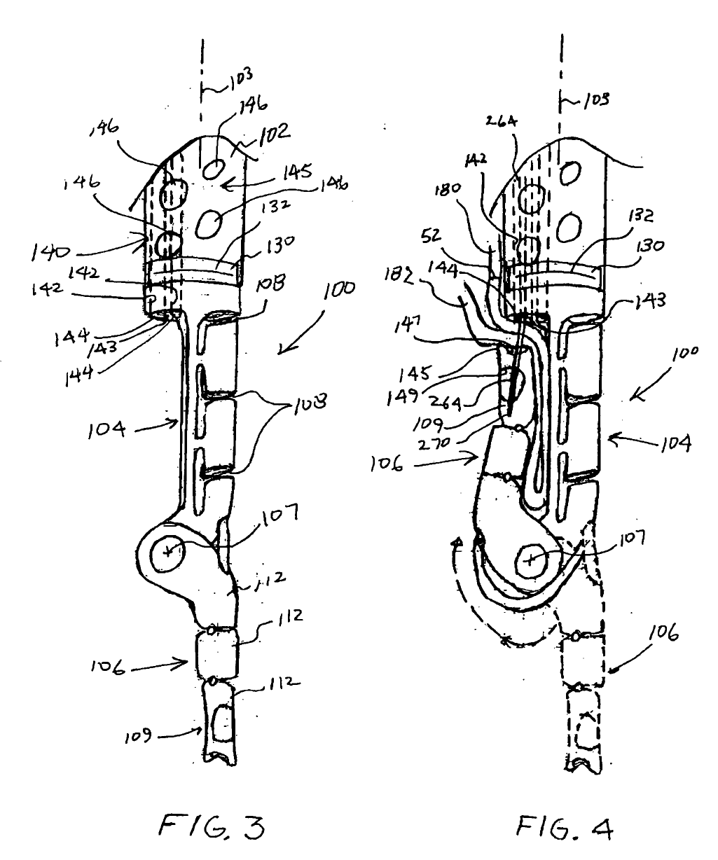

Referring now to FIG. 4, it shows the device 100 forming a flap to restore a GEFV from stomach tissue layers 180 and 182. The tissue layers have been pulled in between the bail 106 and chassis 104 by the gripper and cable (not shown) and the bail has been rotated about pivot 107 to close on the tissue to form a flap portion from tissue layers 180 and 182.

It may be noted in FIG. 4 that the bail 106 has a distal end 145 that includes an opening 147. The opening permits a deployment stylet 264, fed down the guide lumen 142 and through the delivery port 144 to pierce through both tissue layer 180 and 182 during the deployment of a fastener through tissue layers 180 and 182.

More specifically, when the bail 106 is closed on the chassis as shown in FIG. 4, the wall 143 and bail distal end 145 are closely adjacent. The delivery port 144 is aligned with the opening 145 and the tissue layers are bent so that the tissue layers are disposed substantially transverse to the longitudinal axis 103 and presented to the stylet path for fastener deployment. This permits the fastener director guide lumen 142 to direct a fastener along the stylet 264 in a path that is continuously substantially parallel to the longitudinal axis 103. This is in direct contrast to prior arrangements where the guide lumen paths were bent to present a fastener substantially transverse to tissue layers which were not bent. As a result, unlike prior arrangements, the force with which a fastener may be deployed is not diminished. Also, since the fastener is translated over a substantially straighter path, the fastener travel through the guide lumen is rendered much smoother.

Hence, with the bail 106 closed on the chassis 104 with the tissue layers 180 and 182 there between and the delivery port 144 aligned with the opening 147, the stylet may be advanced down the guide lumen 142, through the port 144, through the tissue layers 180 and 182 and through the opening 147 into the distal link 109 of the ball 106. A fastener may now be deployed to fasten the tissue layers 180 and 182 together to maintain the formed flap.

As mentioned above, the end of the stylet 264 is directed into the distal link 109 of the bail 106. The distal link 109 includes a hollow portion which may be referred to as a stylet house that captures and encloses the end of the stylet 264. The stylet distal tip 270 is thus rendered unexposed to surrounding tissue. This safety feature of this embodiment precludes the possibility of the stylet 264 causing injury to surrounding tissue.

It may also be observed that the distal link 109 further includes a side port 149. This side port 149 permits the stylet 264 to be visualized with an endoscope to assist in the proper axial positioning of the stylet 264. It may further serve to permit visualization of the fastener being deployed to both assist in deployment and to confirm fastener deployment.

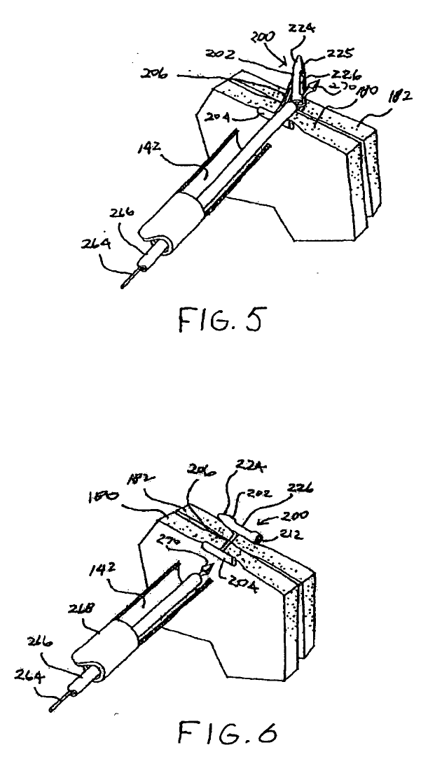

FIGS. 5 and 6 illustrate a manner in which the device 100 of FIGS. 3 and 4 may deploy a fastener 200 through the layers 180 and 182 of stomach tissue. The fastener 200 generally includes a first member 202, a second member 204, and a connecting member 206. As may be noted in FIG. 6, the first member 202 and second member 204 are substantially parallel to each other and substantially perpendicular to the connecting member 206 which connects the first member 202 to the second member 204.

The first member 202 is generally cylindrical or can be any shape. It has a channel 212 that extends therethrough. The through channel 212 is dimensioned to be slidingly received on the tissue piercing deployment wire 264.

The first member 202 includes a pointed tip 224. The tip 224 may be conical and more particularly takes the shape of a truncated cone. The tip can also be shaped to have a cutting edge in order to reduce tissue resistance.

The first member 202 also has a continuous lengthwise slit 225. The slit 225 includes an optional slot 226 that communicates with the through channel 212. The slot 226 has a transverse dimension for more readily enabling receipt of the tissue piercing deployment wire 264 during deployment of the fastener 200.

The fastener member 202 is preferably formed of flexible material. The slit 225 may thus be made larger through separation to allow the deployment wire to be snapped into and released from the through channel 212.

In addition to the fastener 200 and the deployment wire 264, the assembly shown in FIGS. 5 and 6 further includes a pusher 266 and a guide tube 268. The subassembly of the tissue piercing wire 264, fastener 200, and pusher 266 may be guided to its intended location relative to the tissue layers 180 and 182 by the guide tube 268. The tissue piercing wire 264, fastener 200, and the pusher 266 are all initially within the guide tube 268. The guide tube 268 is representative of the fastener deployment guide and to that end, includes the fastener deployment guide lumen 142. The subassembly of the tissue piercing wire 264, fastener 200, and pusher 266 may be guided to its intended location relative to the tissue layers 180 and 182 by the guide lumen 142.

As shown in FIGS. 5 and 6, the tissue piercing wire 264 has a tip 270 helping it pierce the tissue layers 180 and 182 that will form the restored gastroesophageal flap valve. The pusher 266 has pushed the first member 202 of the fastener 200 through the tissue layers 180 and 182 on the tissue piercing wire 264. This may be accomplished by moving the wire 264 and the pusher 266 together.

As may be further noted in FIG. 5, the first member 202 is clearing the wire 264 and tissue layer 182. The tissue piercing wire 264 may now be retracted into the pusher 266 and the tissue piercing wire 264 and pusher 266 may be withdrawn.

FIG. 6 illustrates the fastener 200 in its fully deployed position. It will be noted that the fastener has returned to its original shape. The tissue layers 180 and 182 are fastened together between the first member 202 of the fastener 200 and the second member 204 of the fastener 200. The connecting member 206 extends through the tissue layers 180 and 182. If the additional fastener deployment guides are provided, the foregoing steps for deploying further fasteners may be repeated.

To render the flap uniform about the opening of the orifice into the stomach, it may be necessary to rotate the device 100 and repeat the previously described procedure for forming a further flap portion. When the appearance of the valve flap is satisfactory as viewed through an endoscope, for example, the bail 106 may be moved to a fully opened position as seen, for example, in FIG. 3 and the device 100 may be removed from the stomach and esophagus. This would then complete the procedure of restoring to GEFV.

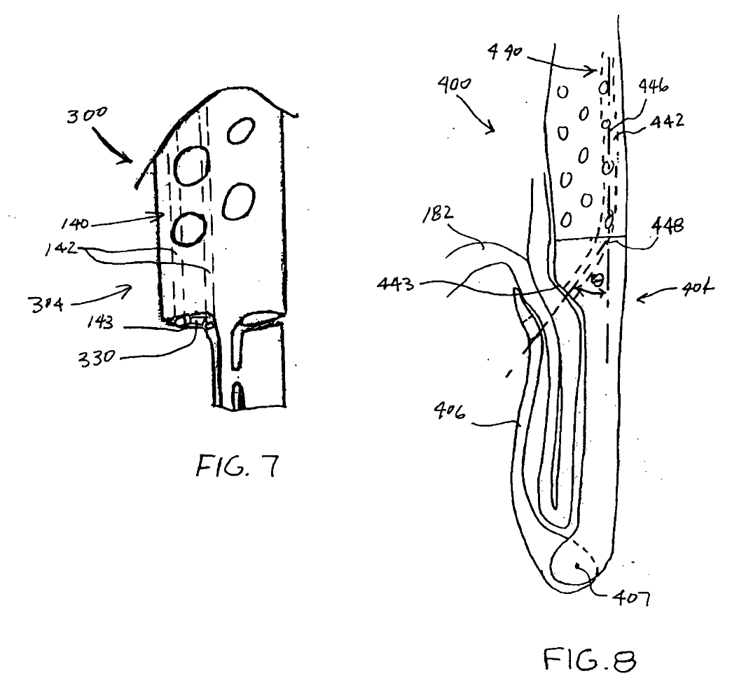

Referring now to FIG. 7, it shows the chassis 304 of another apparatus 300 embodying the present invention. The apparatus 300, as in the previous embodiment, includes the fastener director 140 in the form of fastener directing guide lumens 142. The main difference between the apparatus 300 and the apparatus previously described is that the window for observing tissue is within the wall 143 that is substantially transverse to the fastener director lumens 142. Through this window, the color of the tissue may be readily observed so that it may be known where the Z line is and to make sure that the fasteners are driven into the stomach tissue aboral of the Z line.

FIG. 8 shows yet another embodiment. Here, the apparatus 400 includes a non-linked bail 406 hingedly coupled to its associated chassis 404 at the pivot point 407. The apparatus also includes a fastener director 440 which, as in the previous embodiments, may be one or more fastener guide lumens 442. Here however, it will be noted that the fastener director 440 deviates from a line 446 substantially parallel to the longitudinal axis at point 448 by an angle theta (θ). The lumen 442 then terminates at the wall 443 which is still substantially transverse to the lumen 442.

As may be appreciated by those skilled in the art, the drive force applied to a fastener beyond point 448 will be split into two components, a longitudinal component, and a lateral component. More specifically, the lateral component will vary with the sine of theta and be equal to the total drive force (F) times the sine of theta (F×sine θ). Similarly, the longitudinal component will vary with the cosine of theta and be equal to the total drive force times the cosine of theta (F×cosine θ). When the lateral force becomes greater than the longitudinal force, the fastener will be pushed sideways and the longitudinal force component will become relatively ineffective at driving a fastener through the tissue as required. To make sure that the longitudinal force component is always greater than the lateral force component, F×cosine θ must always be greater than F×sin θ and theta must be no greater than 45 degrees. Hence, at the delivery end of lumen 442, the deviation θ is no greater than 45 degrees. Further, it is preferable the fastener director be devoid of any deviation greater than 45 degrees.

While particular embodiments of the present invention have been shown and described, modifications may be made, and it is thereto intended in the appended claims to cover all such changes and modifications which fall within the true spirit and scope of the invention.

What is claimed is:

1. A transoral gastroesophageal flap valve restoration device comprising:
a longitudinal member, a portion of which is arranged for transoral placement into a stomach;
a tissue shaper carried on the longitudinal member that shapes stomach tissue into a shape; and
a fastener director that directs a fastener into the stomach tissue along a path devoid of any deviation greater than 45 degrees.

2. The device of claim 1, wherein the fastener director comprises a guide lumen that extends from the longitudinal member to the tissue shaper.

3. The device of claim 2 further comprising a deployment wire arranged to guide a fastener through the guide lumen, wherein the deployment wire includes a distal tip, and wherein the tissue shaper is arranged to capture and enclose the distal tip.

4. The device of claim 1, wherein the tissue shaper comprises a pair of hingedly coupled first and second arms for receiving the stomach tissue there between, the second arm including an opening permitting the fastener to be driven through the stomach tissue while being held between the first and second arms.

5. The device of claim 4, wherein the second arm has a distal end and wherein the opening is at the distal end of the second arm.

6. The device of claim 1, wherein the tissue shaper comprises a pair of hingedly coupled first and second arms for receiving the stomach tissue there between, wherein the first arm includes a wall generally transverse to the fastener director and wherein the fastener director directs the fastener through the wall.

7. The device of claim 1 further comprising a window within the wall for observing tissue characteristics.

8. The device of claim 6, wherein the fastener director comprises a guide lumen within the longitudinal member and communicating with the wall.

9. The device of claim 6, wherein the second arm has a distal end, wherein the opening is at the distal end of the second arm, and wherein the distal end is closely adjacent the wall with the opening aligned with the fastener director when a fastener is directed through the stomach tissue.

10. The device of claim 9, wherein the fastener director comprises a guide lumen that extends through the longitudinal member and communicates with the wall.

11. The device of claim 1, comprising a plurality of fastener directors, each fastener director being arranged to direct a fastener into the stomach tissue along a path that is devoid of any deviation greater than 45 degrees.

12. The device of claim 11, wherein each fastener director comprises a guide lumen that extends from the longitudinal member to the tissue shaper.

13. A transoral gastroesophageal flap valve restoration device comprising:
a longitudinal member, a portion of which is arranged for transoral placement into a stomach;
a tissue shaper carried on the longitudinal member that shapes stomach tissue into a shape, the tissue shaper comprising a pair of hingedly coupled first and second arms for receiving the stomach tissue there between; and
a fastener director that directs a fastener into the stomach tissue along a path that is free of bends greater than 45 degrees.

14. The device of claim 13, wherein the fastener director comprises a guide lumen that extends from the longitudinal member to the first member.

15. The device of claim 14 further comprising a deployment wire arranged to guide a fastener through the guide lumen, wherein the deployment wire includes a distal tip, and wherein the tissue shaper is arranged to capture and enclose the distal tip.

16. The device of claim 13, wherein the second arm includes an opening permitting the fastener to be driven through the stomach tissue while being held between the first and second arms.

17. The device of claim 16, wherein the second arm has a distal end and wherein the opening is at the distal end of the second arm.

18. The device of claim 13, wherein the first arm includes a wall generally transverse to the fastener director and wherein the fastener director directs the fastener through the wall.

19. The device of claim 18 further comprising a window within the wall for observing tissue characteristics.

20. The device of claim 18, wherein the fastener director comprises a guide lumen within the longitudinal member and communicating with the wall.

21. The device of claim 18, wherein the second arm has a distal end, wherein the opening is at the distal end of the second arm, and wherein the distal end is closely adjacent the wall with the opening aligned with the fastener director when a fastener is directed through the stomach tissue.

22. The device of claim 21, wherein the fastener director comprises a guide lumen that extends through the longitudinal member and communicates with the wall.

23. The device of claim 13, comprising a plurality of fastener directors, each fastener director being arranged to direct a fastener into the stomach tissue along a path devoid of any deviation greater than 45 degrees.

24. The device of claim 23, wherein each fastener director comprises a guide lumen that extends from the longitudinal member to the first member.

25. An assembly for transoral gastroesophageal flap valve restoration comprising:
a longitudinal member, a portion of which is arranged for transoral placement into a stomach;
a tissue shaper carried on the longitudinal member that shapes stomach tissue into a shape;
a fastener that maintains the stomach tissue in the shape; and
a fastener director that directs the fastener into the stomach tissue along a path that is devoid of any deviation greater than 45 degrees.

26. The assembly of claim 25, wherein the fastener director comprises a guide lumen that extends from the longitudinal member to the tissue shaper.

27. The assembly of claim 26, further comprising a deployment wire arranged to slidingly receive the fastener and guide the fastener down the guide lumen and into the stomach tissue.

28. The assembly of claim 27 wherein the deployment wire includes a distal tip end and wherein the tissue shaper is configured to receive and enclose the deployment wire tip end.

29. The assembly of claim 27, further comprising a pusher that pushes the fastener along the deployment wire down the guide lumen.

30. The assembly of claim 27, wherein the fastener comprises first and second members having first and second ends, and a connecting member fixed to each of the first and second members intermediate the first and second ends and extending between the first and second members, wherein the first and second members are separated by the connecting member, and wherein one of the first and second members has a through channel for being received on the deployment wire.

31. The assembly of claim 25, wherein the tissue shaper comprises a pair of hingedly coupled first and second arms for receiving the stomach tissue there between, the second arm including an opening permitting the fastener to be driven through the stomach tissue while being held between the first and second arms.

32. The assembly of claim 31, wherein the second arm has a distal end and wherein the opening is at the distal end of the second arm.

33. The assembly of claim 25, wherein the tissue shaper comprises a pair of hingedly coupled first and second arms for receiving the stomach tissue there between, wherein the first arm includes a wall generally transverse to the fastener director and wherein the fastener director directs the fastener through the wall.

34. The assembly of claim 33 further comprising a window within the wall for observing tissue characteristics.

35. The assembly of claim 33, wherein the fastener director comprises a guide lumen within the longitudinal member and communicating with the wall.

36. The assembly of claim 33, wherein the second arm has a distal end, wherein the opening is at the distal end of the second arm, and wherein the distal end is closely adjacent the wall with the opening aligned with the fastener director when a fastener is directed through the stomach tissue.

37. The assembly of claim 36, wherein the fastener director comprises a guide lumen that extends through the longitudinal member and communicates with the wall.

38. The assembly of claim 25, comprising a plurality of fastener directors, each fastener director being arranged to direct a fastener into the stomach tissue along a path that is devoid of any deviation greater than 45 degrees.

39. The assembly of claim 38, wherein each fastener director comprises a guide lumen that extends from the longitudinal member to the tissue shaper.

40. A transoral gastroesophageal flap valve restoration device comprising:
a longitudinal member, a portion of which is arranged for transoral placement into a stomach;
a tissue shaper carried on the longitudinal member that shapes stomach tissue into a shape; and
a fastener director that directs a fastener along a fastener deployment wire and a path into the stomach tissue, the fastener deployment wire having a distal tip end,
the tissue shaper being configured to receive and enclose the deployment wire tip end.

41. The device of claim 40, wherein the path is devoid of any deviation greater than 45 degrees.

42. The device of claim 40, wherein the fastener director comprises a guide lumen that extends from the longitudinal member to the tissue shaper.

43. The device of claim 40, wherein the tissue shaper comprises a pair of hingedly coupled first and second arms for receiving the stomach tissue there between and wherein the second arm is configured to receive and enclose the deployment wire tip end.

44. The device of claim 43, wherein the second arm has a distal end and wherein the distal end of the second end is configured to receive and enclose the deployment wire tip end.

45. The device of claim 40, wherein the fastener director comprises a guide lumen within the longitudinal member.
